(19) United States
(12) Patent Application Publication
(10) Pub. No.: US 2026/0331299 A1
(43) Pub. Date: Oct. 22, 2026

(54) SYSTEMS AND METHODS FOR MODELLING PHYSIOLOGIC FUNCTION USING A COMBINATION OF MODELS OF VARYING DETAIL

(71) Applicant: THE BOARD OF TRUSTEES OF THE LELAND STANFORD JUNIOR UNIVERSITY, Stanford, CA (US)

(72) Inventors: Ryan Leonard Spilker, Arlington, MA (US); Charles Anthony TAYLOR, JR., Atherton, CA (US)

(21) Appl. No.: 19/427,319

(22) Filed: Dec. 19, 2025

Related U.S. Application Data
(63) Continuation of application No. 17/394,591, filed on Aug. 5, 2021, now Pat. No. 12,525,361.
Application No. 17/394,591 is a continuation of application No. 13/464,452, filed on May 4, 2012, now Pat. No. 11,107,587.
Application No. 13/464,452 is a continuation of application No. 12/219,398, filed on Jul. 21, 2008, now Pat. No. 8,200,466.

(51) Int. Cl. G16H 50/50; G06F 30/20; G06F 111/10; G06T 17/20; G06T 19/20; G16H 10/60; G16H 30/40

(52) U.S. Cl. CPC G16H 50/50; G06F 30/20; G06T 17/20; G06T 19/20; G16H 10/60; G16H 30/40; G06F 2111/10; G06T 2210/24; G06T 2210/41

(57) ABSTRACT

Computational methods are used to create cardiovascular simulations having desired hemodynamic features. Cardiovascular modeling methods produce descriptions of blood flow and pressure in the heart and vascular networks. Numerical methods optimize and solve nonlinear equations to find parameter values that result in desired hemodynamic characteristics including related flow and pressure at various locations in the cardiovascular system, movements of soft tissues, and changes for different physiological states. The modeling methods employ simplified models to approximate the behavior of more complex models with the goal of to reducing computational expense. The user describes the desired features of the final cardiovascular simulation and provides minimal input, and the system automates the search for the final patient-specific cardiovascular model.

### RELATED APPLICATIONS

This application continuation of and claims the benefit of priority to U.S. application Ser. No. 17/394,591, filed on Aug. 5, 2021, which is a continuation of and claims the benefit of priority to U.S. application Ser. No. 13/464,452, filed on May 4, 2012, now U.S. Pat. No. 11,107,587, which is a continuation of U.S. application Ser. No. 12/219,398, filed on Jul. 21, 2008, now U.S. Pat. No. 8,200,466, the entireties of all of which are incorporated herein by reference.

### STATEMENT REGARDING FEDERAL RIGHTS

This Invention Was Made in Part With Government Support Under Grant Number 0205741 awarded by the National Science Foundation. The Government has certain rights in the invention.

### BACKGROUND OF THE INVENTION

### 1. Field of the Invention

This invention relates generally to computer-assisted modeling and planning systems and in particular to a computer-assisted human anatomic and physiologic modeling system used to predict outcomes of medical intervention and further to predict changes in physiologic function under various states, stresses, and environments and still further to generate data for disease research or medical device design.

### 2. Discussion of Prior Art

Disclosed in Kamm et al., U.S. Pat. No. 6,117,087, is a method and apparatus for deriving a physiological description and clinically-useful data regarding the cardiovascular system of an individual subject. The method includes obtaining a measurement sample associated with cardiovascular flow and utilizing a model, which may be distributed and/or non-linear to derive a description and data. The model generates and uses functions of source parameters and may, in an embodiment, match measurement samples against a library of stored, predicted samples. A best-matching, predicted sample may then be associated with a measurement sample. An apparatus is provided that, according to an embodiment, includes an input for obtaining a measurement sample, a processor to derive the description and data, and an output. The apparatus may also include a digital storage medium to store a library of predicted samples.

Disclosed in Taylor et al., U.S. Pat. No. 6,236,878, is a method for predictive modeling of human anatomy and physiologic function for planning medical interventions on at least one portion of a body with the goals of improving the outcome of the medical intervention and reducing the risks associated with medical intervention. The method comprises the steps of generation of multi-dimensional continuous geometric models of human anatomy, the generation of models of physiologic functions, the integration of the multi-dimensional continuous geometric human anatomy models with the physiologic functional models, and the use of the integrated models to predict the outcome of medical interventions. Also disclosed is a method for the integration of multi-dimensional continuous geometric models of human anatomy and models of physiologic functions to evaluate and predict changes in physiologic function in various functional states, stresses and environments and a method for generating data for disease research.

A method and apparatus for modeling circulation in a living subject is disclosed in Charbel et al, U.S. Pat. No. 7,191,110. The method includes the steps of developing a model for living subjects in general and correcting the model to substantially conform to the overall cerebral physiology of the living subject. The method further includes the step of calculating a cerebral flow of the living subject based upon the corrected model and a selected cerebral blood flow perturbation.

The paper by Spilker et al., Ann Biomed Eng (2007) 35:546-559. DOI 10.1007/s10439-006-9240-3, demonstrates the tuning of a model of pulmonary arterial hemodynamics with morphometry-based impedance outlet boundary conditions. A reduced-order model using steady flow was used to help initialize the tuning of a mean flow fraction and mean pressure of a more detailed model.

The paper by Ellwein et al., Cardiovasc Eng (2008) 8:73-87. DOI 10.1007/s10558-007-9050-8, describes a search for parameter values for unsteady cardiovascular simulations. This work involved tuning lumped-parameter models and, more specifically, models that were reduced to systems of ordinary differential equations (ODE's), which were solved with numerical methods for temporal integration of ODE's. An effort was made to identify the parameters to which the cardiovascular model was most sensitive.

This invention incorporates tuning of reduced-order models of unsteady cardiovascular dynamics and, in addition, reduced-order models that can be solved more quickly using Fourier analysis. In addition, this invention provides the framework for tuning features of time-varying hemodynamic simulations that allows the user to choose either a single objective function that combines many objectives, as in the work of Ellwein et al., or a set of objectives of the same size as the set of parameters so that a solution of a nonlinear system can be sought. This novel use of a limited set of features of the hemodynamic simulations as objectives can give the user control over the most important aspects of the simulation and may save computational energy.

A key difference between this invention and Kamm et al. U.S. Pat. No. 6,117,087, Taylor et al. U.S. Pat. No. 6,236,878, Charbel et al. U.S. Pat. No. 7,191,110, and the work of Ellwein et al. is this invention's use of these less detailed models in the process of tuning hemodynamic simulations that are significantly more computationally expensive. This difference makes this invention most valuable when implemented in software for modeling blood flow in three dimensions for the purposes of intervention planning. disease research and medical device design and evaluation. Previous work does not describe time-varying hemodynamic models of various degrees of complexity and their connection to one another. This aspect of this invention, along with the automation of the tuning process, will save users and computers significant time and effort.

### SUMMARY OF THE INVENTION

Cardiovascular models have the potential to improve our understanding of the mechanics of the circulatory system in healthy and diseased states and to provide quantitative predictions of the outcomes of various interventions to aid in planning treatments. In order to serve this purpose, hemodynamic simulations must have the ability to faithfully represent a patient's circulation prior to treatment. Recent advances in the fidelity of hemodynamic simulations include the use of anatomic models constructed from medical image data, deformation of vascular walls, and improved representation of downstream vascular beds using appropriate outlet boundary conditions. This invention provides a method for employing these capabilities, enabling the systematic tuning of a hemodynamic simulation's parameters such that its hemodynamic characteristics match more than simply patient-specific vascular geometry, inlet flow, mean flow rates, and mean pressures.

### BRIEF DESCRIPTION OF THE DRAWING

FIG. 1 is a flow chart illustrating an embodiment of the method of the present invention for tuning patient-specific hemodynamic simulations.

### DETAILED DESCRIPTION

The present invention applies computational methods to create cardiovascular simulations having desired hemodynamic features. A variety of cardiovascular modeling methods can be used to produce descriptions of time-varying blood flow and pressure in the heart and vascular networks. Numerical methods for optimization and solution of nonlinear systems of equations are used to find values for the parameters used in such models that result in simulations that match desired hemodynamic characteristics. These characteristics will often be those of the cardiovascular system of a particular subject. A variety of modeling methods are used for the same cardiovascular system such that more simplified models can be used to approximate the behavior of more detailed and complex models, which may lead to reduced computational expense. A system is created from these methods such that the user describes the desired features of the final cardiovascular simulation and provides minimal input, and the system automates the search for the final cardiovascular model.

Embodiments of this invention address cardiovascular models with a variety of parameters including, but not limited to, resistances, impedances, compliances, inductances, cardiac chamber elastances, material parameters of tissues, vascular network structures, and changes in these parameters with changes in physiological, disease, and interventional states. Also included are relationships between the various parameters of the models, including time constants defined by combinations of resistances and capacitors in lumped-parameter regions of cardiovascular models.

The desired features, called objectives, of the cardiovascular models addressed by embodiments of this invention include, but are not limited to, features of pressure waveforms, features of flow waveforms, features of time-varying velocity fields, features of transport, features of cardiac volume, features of tissue movements, and variations in these features with changes in physiological, disease, and interventional states. Relevant features of a time-varying quantity include the maximum, minimum, median, mean value over a certain portion of the cardiac cycle, and values at particular times.

The computational expense of solving the mathematical equations associated with a model of the cardiovascular system is generally related to the model's level of detail. The cost of solving these equations for several sets of values of a highly detailed model's parameters can be prohibitive. This motivates the approximation of a highly detailed model's behavior in a model that can be evaluated less expensively. For example, a numerical solution of the mechanics of blood flow and vascular motion in a three-dimensional arterial network with inlet boundary conditions representing the movement of the heart, outlet boundary conditions involving lumped-parameter models that approximate the behavior of downstream vascular networks, and deformable vessel walls has a high computational cost. This model's flow waveforms may be approximated, in an embodiment of this invention, by a lumped-parameter model that includes the inlet and outlet boundary conditions of the complex model and lumped-parameter models that approximate the impedances of the flow pathways from the inlet to the outlets, called intrinsic impedances. The reduced-order model allows testing many values for the lumped parameters of the inlet and outlet boundary conditions with low computational cost and can be used to help find a combination of these values that produces desired hemodynamic features in the highly detailed model. If the successful parameters of the lumped-parameter model do not meet the same success in the highly detailed model, the lumped-parameter model can be adjusted to contain more accurate intrinsic impedances. Other embodiments reduce the intrinsic impedances of the highly detailed model to impedance spectra in the reduced-order model.

Some embodiments of this method rely on less detailed models only to provide an initial estimate of the parameters of the more detailed models that will result in the desired hemodynamic features. Some embodiments use the less detailed models to determine the variations in cardiovascular model outputs with variations in parameter values.

The application of a preferred embodiment of this invention to the tuning of a specific model of blood flow in the abdominal aorta provides an illustrative example of the method disclosed herein. Patient-specific flow waveforms are measured in two locations, one proximal to the celiac bifurcation and one distal to the renal arteries. Maximum and minimum blood pressures are recorded. A detailed model is chosen, consisting of a geometric model of the abdominal aorta and its largest branch vessels, created from medical image data, a highly refined mesh of the geometric model on which the incompressible Navier-Stokes equations are solved in three-dimensions using a finite element method. Vessel wall deformations are modeled. Measured flow is applied at the inlet, and outlet boundary conditions are impedances of three-element windkessel lumped-parameter models. The objectives are the desired maximum, minimum, and median of the pressure waveform, the amplitude and diastolic mean value of the measured infrarenal flow waveform, and the mean flow through the infrarenal plane. A set of parameters is chosen by varying several sets of parameters and observing the resulting changes in flow and pressure waveforms. The model parameters chosen to be tuned are the total resistance of the outlets proximal to the infrarenal plane, the total resistance of the distal outlets, and mean values and ratios of the values of the following two quantities for the three-element windkessels proximal and distal to the plane: the ratio of the proximal resistor to the total resistance and the time constant, defined by the product of the capacitance and the distal resistance. Resistances of the outlet vessels are determined by the total resistance of the region to which they belong and the approximate mean flow fractions found in the literature. The tuning of the boundary condition parameters is formulated as a system of six nonlinear equations in six unknowns, seeking a root where the simulated and measured hemodynamic conditions match. This nonlinear system is solved using a quasi-Newton method where each function evaluation requires one three-dimensional simulation. The Jacobian of this system is updated using a version of Broyden's method in which attention is paid to scaling of the different parameters to distribute the secant updates appropriately. The objectives are evaluated first using a reduced-order model consisting of the impedances of the three-element windkessels in parallel. This lumped-parameter model is solved to determine the initial boundary condition parameters for the three-dimensional model. Three-dimensional simulations are run on a coarse initial mesh with a boundary layer at the vessel wall. When a solution of this model is found, the mesh is further refined, more boundary layers are added, and the tuning continues until the simulation on an adequately refined mesh matches the objectives to a desired tolerance. This example illustrates the tuning of parameters that are related to resistances and capacitances. It also illustrates the use of a lumped-parameter model as a reduced-order model for the more detailed cardiovascular model.

This invention employs a range of levels of detail in cardiovascular models. One possible increase in the level of detail is a change from a model using prescribed flow in a blood vessel to a model using an elastance-based model of a cardiac chamber. This allows the prediction of the reaction of the heart to changes in afterload. Another possible increase in the level of detail comes with a change from a model that can be described by a system of ordinary differential equations that can be solved with Fourier analysis to a model that can be described by a system of ordinary differential equations that must be solved with numerical methods for temporal integration. This may occur when a cardiovascular model with an elastance-based heart model is desired. Increases in the level of detail of cardiovascular models also are associated with a change from a model assuming rigid vascular walls to a model assuming more realistic representations of these walls and, in numerical solutions of partial differential equations governing the cardiovascular system, increasingly refined computational meshes and increasingly complete polynomial bases.

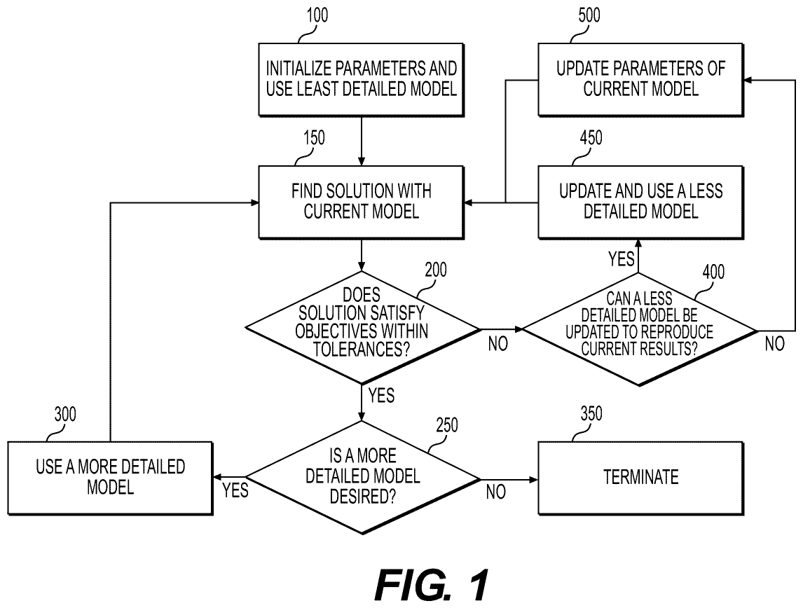

The steps in a preferred embodiment of the method for tuning patient-specific hemodynamic simulations are shown in FIG. 1. The parameters for the least detailed model of the cardiovascular system are initialized 100. This step can be aided by prior knowledge of ranges of values for each parameter of such a model. Next, the solution of the mathematical equations governing the current model is sought 150. This includes the determination of the change in the model's features with changes in the model's parameters. A first determination 200 is made if the solution satisfies the objectives within tolerances. If it does, then a second determination 250 is made if a more detailed. model is desired. If one is not, then the method has reached a final solution 350. If the second determination 250 is that a more complex model is needed, then a second model 300 is used wherein the level of detail of the second model is increased. The more complex second model 300 is then solved 150 and the process repeated. If the first determination 200 does not satisfy the objectives within tolerances, a third determination 400 is made if a less detailed model can be updated to reproduce current results. If it can, a third model with a reduced level of detail is updated 450 such that it reproduces, within a reasonable tolerance, the important aspects of the more detailed model's results. The less detailed model is then solved 150. If the third determination 400 is that a less detailed model cannot be updated to reproduce current results, then a new model 500 is created by updating the parameters of the current model 150, and the method is repeated until the method has reached a final solution 350.

In a related embodiment of this invention, a less detailed model that approximates the results of a more detailed cardiovascular model is produced as the final result of the tuning procedure. Less detailed models can be used to succinctly characterize cardiovascular systems and in applications requiring rapid simulations, such as the study of relationships between a model's parameters and its behavior.

Some embodiments of this invention involve evaluation of the appropriateness of parameter sets for achieving objectives of cardiovascular dynamics. Certain sets of parameters are associated with better performance of tuning procedures. Parameter sets to which all components of the objective functions are adequately sensitive are desired. Evaluation of various parameter sets can be performed quickly using representative models with less detail.

This invention solves the problem of making cardiovascular simulations match desired hemodynamic characteristics without excessive and ad hoc user interactions. Systematic tuning is enabled by the formulation of one of several problems. A single objective function can be chosen to summarize the differences between the desired and current features of the cardiovascular model. Embodiments with such a cost function employ optimization methods that minimize this difference. In other embodiments having the same number of parameters as objectives, a solution of a system of nonlinear equations is sought. In other embodiments, a solution of a nonlinear least-squares problem is sought. Numerical methods for solving each of these problems are chosen such that the number of function evaluations is kept low. Finite-difference evaluations of Hessian and Jacobian matrices are reduced, in some embodiments, by use of secant updates. Estimates of these matrices are created, in some embodiments, using evaluations of less detailed models.

Highly detailed cardiovascular models may require the use of lumped parameters to represent regions of the cardiovascular system. To aid in the process of choosing parameters for these models without repeated calculation, an embodiment of this invention determines relationships between lumped parameter values and parameters of more detailed distributed cardiovascular models by approximating these more detailed models with lumped-parameter models. The derived relationships can be stored and used by other embodiments to save computational effort.

Several embodiments of the present invention involve analysis of variations of the parameters of cardiovascular models. Patient-specific measurements have natural variations and, when associated with tuned sets of parameters, can provide succinct information about variability of the individual's cardiovascular system. Members of a class of patients can likewise be analyzed for variability within that class of patients. Related embodiments use such information on variability to analyze ranges of potential outcomes of treatments.

The present invention improves upon existing methods by providing a framework for creating state-of-the-art, detailed cardiovascular models that have desired hemodynamic characteristics with methods that automate the procedure and seek to minimize the computational effort. Existing methods use simplified mathematical models or seek to match only temporal mean values of time-varying quantities. Automation of the process allows users to request features of pressure, flow, vascular motion, cardiac motion, and other physiological data and, after providing anatomic and physiological information for the desired cardiovascular model, take no further action while the system produces a mathematical vascular model with the requested results. This capability enables the creation of patient-specific simulations and predictions of the cardiovascular response to various treatments.

Variations can be created from the general approach defined herein. A variety of cardiovascular modeling methods can be used to create the models with various levels of detail and simplifying assumptions. A variety of numerical methods can be used to tune parameters of the cardiovascular models. A variety of sets of hemodynamic objectives and parameters can also be chosen. A variety of algorithms can be chosen to link models of various levels of detail.

In surgical treatment planning, this invention can be used to create cardiovascular models that match hemodynamic features of a specific patient before predicting the results of a variety of surgical options. In medical device development, this invention can be used to create cardiovascular models that match a variety of possible physiological situations to use in simulating the effects of devices on blood flow and pressure or the forces acting on devices that might affect their short-term and long-term safety and efficacy. In cardiovascular tissue engineering, this invention can be used to create cardiovascular models with realistic pressure, flow, and deformation to develop an understanding of the biomechanical environment of tissues.

The present invention allows the user to predict hemodynamic results of interventions without expensive biological experiments thereby reducing user interaction time and computational expense to create realistic cardiovascular models.

1-19. (canceled)

20. A computer-implemented method for generating a reduced-order model of a cardiovascular system, comprising:
obtaining a model of at least a portion of the cardiovascular system;
segmenting the model into a plurality of regions of the cardiovascular system;
determining a cardiovascular parameter of at least one region of the plurality of regions; and
for each region of the at least one region:
mapping the cardiovascular parameter to one of a plurality of stored cardiovascular parameters, wherein each of the plurality of stored cardiovascular parameters has a predetermined relationship with one or more lumped parameters; and
replacing the region with the one or more lumped parameters.

21. The computer-implemented method of claim 20, wherein the predetermined relationship between the one or more lumped parameters and each parameter of the plurality of stored cardiovascular parameters is based on a comparison between the one or more lumped parameters and an approximation of a cardiovascular model having the parameter using a lumped parameter model.

22. The computer-implemented method of claim 20, wherein the at least one region includes every region of the model, such that every region of the model is replaced with one or more corresponding lumped parameters.

23. The computer-implemented method of claim 20, wherein obtaining the model includes:
receiving patient-specific image data associated with the cardiovascular system; and
generating the model based on the received patient-specific image data, the generating including representing at least one inlet and at least one outlet of blood flow of the portion of the cardiovascular system represented in the model.

24. The computer-implemented method of claim 20, wherein the one or more lumped parameters are computationally faster than the region replaced.

25. The computer-implemented method of claim 20, wherein the one or more lumped parameters approximate at least one intrinsic impedance of the region of the cardiovascular system.

26. The computer-implemented method of claim 20, wherein the model is a three-dimensional model.

27. The computer-implemented method of claim 20, further comprising:
determining at least one blood flow characteristic of the cardiovascular system using the model with the at least one region replaced by the one or more lumped parameters.

28. A system for generating a reduced-order model of a cardiovascular system, comprising:
at least one memory storing instructions; and
at least one processor operatively connected to the at least one memory and configured to execute the instructions to perform operations, comprising:
obtaining a model of at least a portion of the cardiovascular system;
segmenting the model into a plurality of regions of the cardiovascular system;
determining a cardiovascular parameter of at least one region of the plurality of regions; and
for each region of the at least one region:
mapping the cardiovascular parameter to one of a plurality of stored cardiovascular parameters, wherein each of the plurality of stored cardiovascular parameters has a predetermined relationship with one or more lumped parameters; and
replacing the region with the one or more lumped parameters.

29. The system of claim 28, wherein the predetermined relationship between the one or more lumped parameters and each parameter of the plurality of stored cardiovascular parameters is based on a comparison between the one or more lumped parameters and an approximation of a cardiovascular model having the parameter using a lumped parameter model.

30. The system of claim 28, wherein the at least one region includes every region of the model, such that every region of the model is replaced with one or more corresponding lumped parameters.

31. The system of claim 28, wherein obtaining the model includes:
receiving patient-specific image data associated with the cardiovascular system; and
generating the model based on the received patient-specific image data, the generating including representing at least one inlet and at least one outlet of blood flow of the portion of the cardiovascular system represented in the model.

32. The system of claim 28, wherein:
the one or more lumped parameters are computationally faster than the region replaced; and
the one or more lumped parameters approximate at least one intrinsic impedance of the region of the cardiovascular system.

33. The system of claim 28, wherein the model is a three-dimensional model.

34. The system of claim 28, wherein the operations further include:
determining at least one blood flow characteristic of the cardiovascular system using the model with the at least one region replaced by the one or more lumped parameters.

35. A non-transitory computer-readable medium comprising instructions for generating a reduced-order model of a cardiovascular system, the instructions operable by one or more processors to perform operations, including:
obtaining a model of at least a portion of the cardiovascular system;
segmenting the model into a plurality of regions of the cardiovascular system;
determining a cardiovascular parameter of at least one region of the plurality of regions; and
for each region of the at least one region:
mapping the cardiovascular parameter to one of a plurality of stored cardiovascular parameters, wherein each of the plurality of stored cardiovascular parameters has a predetermined relationship with one or more lumped parameters; and
replacing the region with the one or more lumped parameters.

36. The non-transitory computer-readable medium of claim 35, wherein the predetermined relationship between the one or more lumped parameters and each parameter of the plurality of stored cardiovascular parameters is based on a comparison between the one or more lumped parameters and an approximation of a cardiovascular model having the parameter using a lumped parameter model.

37. The non-transitory computer-readable medium of claim 35, wherein the at least one region includes every region of the model, such that every region of the model is replaced with one or more corresponding lumped parameters.

38. The non-transitory computer-readable medium of claim 35, wherein obtaining the model includes:
receiving patient-specific image data associated with the cardiovascular system; and
generating the model based on the received patient-specific image data, the generating including representing at least one inlet and at least one outlet of blood flow of the portion of the cardiovascular system represented in the model.

39. The non-transitory computer-readable medium of claim 35, wherein the operations further include:
determining at least one blood flow characteristic of the cardiovascular system using the model with the at least one region replaced by the one or more lumped parameters.
